(19) United States
(12) Patent Application Publication
(10) Pub. No.: US 2007/0238764 A1
(43) Pub. Date: Oct. 11, 2007

(54) Use of Sphingoid Base Associated with Nicotinic Acid or a Nicotinic Acid Amide in the Form of Depigmentation Agent

(76) Inventors: Jean-Claude Allart, Le Touquet (FR); Jean-Marie Lefevre, Amiens (FR); Jacques Peyrot, Clemont-Ferrand (FR)

Correspondence Address: BUCHANAN, INGERSOLL & ROONEY PC, POST OFFICE BOX 1404, ALEXANDRIA, VA, 22313-1404, US

(21) Appl. No.: 11/547,846

(22) PCT Filed: Apr. 8, 2005
(86) PCT No.: PCT/FR05/00864

§ 371 Date: Oct. 6, 2006

(30) Foreign Application Priority Data
Apr. 9, 2004 (FR) 0403740

(51) Int. Cl. A61P 17/00; A61K 31/133; A61K 31/455

(52) U.S. Cl. 514/356; 568/704

(57) ABSTRACT

The invention relates to using a composition comprising an association of at least one type of nicotinic acid or nicotinic acid amide and at least one type of sphingoid base in the form of a depigmentation agent. The use of the inventive composition makes it possible simultaneously to promote skin lightening and to control a local skin hyperpigmentation of any nature and origin. Methods for an associated cosmetic treatment and the use of an association of at least one type of nicotinic acid or nicotinic acid amide and at least one type of sphingoid base for preparing a dermatological preparation for controlling hyperpigmentation are also disclosed.

The present invention relates to a novel use of a composition comprising a combination of at least nicotinic acid or a nicotinic acid amide and at least one sphingoid base.

### PRIOR ART

Skin depigmentation may be desired under various circumstances, either for overall lightening of the skin, or for eliminating or reducing marks derived from local problems with pigmentation.

Skin pigmentation is determined by the presence of melanin in the epidermis and the dermis. Melanin is produced by melanocytes located mainly in the basal layer. These melanocytes send out dendritic extensions just about everywhere, between the keratocytes. The presence of an enzyme, tyrosinase, is required in order to produce melanin. This biosynthesis, also called melanogenesis, is a complex process which is now relatively well known.

Thus, pigmentation is normally uniform. However, pigmentation can be locally excessive, which is commonly referred to as hyperpigmentation. Skin hyperpigmentation can include skin deficiencies or disorders comprising freckles, senile lentigo, melasma, age spots, pigmentation due to sunburn, post-inflammatory hyperpigmentation due to abrasions, burns, wounds, insect bites, dermatitis, and other small pigmented local lesions.

A number of cosmetic treatment methods for hyperpigmentation exist, but none is completely satisfactory. Methods for scrubbing, including chemical scrubbing, in fact exist, or alternatively methods which have an effect on melanogenesis, using depigmenting agents.

The depigmenting agents commonly used act directly on the vitality of epidermal melanocytes where melanogenesis takes place and/or interfere with one of the steps of melanin biosynthesis either by inhibiting one of the enzymes involved in melanogenesis, or by intercalating as a structural analog of one of the chemical compounds of the melanin synthesis chain, which chain can then be blocked, and thus ensure depigmentation.

Depigmenting agents can be divided up into several groups:
phenolic compounds, which comprise in particular the following compounds:
   hydroquinone and ethers thereof such as monobenzyl ether,
   4-methoxyphenol,
   4-isopropylcatechol,
   4-hydroxyanisole, and
   N-acetyl-4-S-cystaminylphenol,
nonphenolic compounds, which comprise in particular the following compounds:
   corticosteroids,
   tretinoin,
   azelaic acid,
   N-acetylcysteine (NAC),
   ascorbic acid and derivatives such as L-ascorbyl-2-phosphate, and
   kojic acid,
combinations of these compounds, among which mention may be made of the Kligman formulation, the Pathak formulation or the Westerhof formulation.

In addition, nicotinamide or niacinamide, also called vitamin PP, vitamin B3 or alternatively niacin, is also known for its depigmenting properties. Application WO 99/47114 also describes compositions comprising essentially nicotinic acid or nicotinamide and also an intermediary or precursor of ceramides, used only for moisturization of the skin and not for depigmentation thereof.

Moreover, it is known that sphingoid bases such as phyto-sphingosine and sphingosine, which are ceramide precursors, are present in human skin, and studies have shown that these molecules have inhibitory properties on protein kinase C, and appear to be involved in epidermal keratinocyte differentiation. It has also been observed that sphingosines present in the stratum corneum and other layers of the epidermis inhibit the growth of certain undesirable microorganisms.

Various publications describe the use of sphingosine and of phytosphingosine in cosmetic and dermatological compositions. For example, patent application WO 95/03028 describes sphingosine, used in a composition at a pH close to 5, for reducing the irritant effect of certain alpha-hydroxy acids. Patent EP 940.140 describes a cosmetic composition combining alpha-hydroxy acids and ceramides or ceramide precursors such as phytosphingosine. However, to date, sphingoid bases have not been found to be effective as depigmenting agents.

Application EP 919.226 relates to a phytosphingosine salicylate-based cosmetic composition which may have anti-acne, anti-wrinkle and skin-lightening properties. The composition may also contain various usual ingredients of the art, such as vitamins A, C and E, used according to their well-known properties.

It is, moreover, known from patent application WO 02/085362 that a composition comprising essentially nicotinic acid or a nicotinic acid amide and a sphingoid base can be used for the treatment of keratinization conditions in human or animal dermatology. In particular, this combination, and in particular that of a nicotinic acid amide such as vitamin PP and of a sphingoid base, is described as being of use in the treatment of acne, in particular common acne, and of atopic dermatitis.

Consequently, there is an increased need in the cosmetological field to find alternative methods for treating hyperpigmentation, and therefore to identify compounds having depigmenting properties.

The studies carried out by the applicant have shown a synergistic effect on depigmentation by combining nicotinic acid or a nicotinic acid amide such as vitamin PP, and a sphingoid base within the same composition.

### THE PRESENT INVENTION

A subject of the invention is therefore the use of a composition comprising a combination of at least nicotinic acid or a nicotinic acid amide and of at least one sphingoid base, as a depigmenting agent.

A subject of the invention is also a method for depigmenting the skin, consisting in applying to the exposed areas a composition comprising a combination of at least nicotinic acid or a nicotinic acid amide and of at least one sphingoid base.

In the literature, a compound which can be used in the cosmetics field is sometimes described as having “lightening properties” for the skin ; in the context of the present invention, the “depigmenting properties” also comprise these “lightening properties” in addition to the properties for controlling hyperpigmentation.

Consequently, a subject of the invention is more particularly the use of a combination of at least nicotinic acid or a nicotinic acid amide and of at least one sphingoid base, for promoting lightening of the skin, and also the associated cosmetic treatment method.

A subject of the invention is therefore also more particularly the use of a combination of at least nicotinic acid or a nicotinic acid amide and of at least one sphingoid base, for controlling skin hyperpigmentation, and also the associated cosmetic treatment method.

In other words, the invention also relates to a method for cosmetic treatment of the skin, intended to reduce, eliminate or avoid pigmentation spots, by providing a depigmenting or lightening effect, consisting in applying to the exposed areas a composition comprising a combination of at least nicotinic acid or a nicotinic acid amide and of at least one sphingoid base.

Finally, a subject of the invention is the use of a combination of at least nicotinic acid or a nicotinic acid amide and of at least one sphingoid base, for preparing a dermatological composition intended to control hyperpigmentation.

The nicotinic acid amide can preferably be nicotinamide or vitamin PP. The sphingoid base can be chosen from sphingosine, sphinganine, phytosphingosine, salicyloyl phytosphingosine, phytosphingosine salicylate, tetracetyl-phytosphingosine, N-acetylphytosphingosine and phyto-sphingosine hydrochloride. According to the invention, the preferred sphingoid base is phytosphingosine, salicyloyl phytosphingosine (N-salicyloyl phytosphingosine) or phytosphingosine hydrochloride.

The nicotinic acid, or the nicotinic acid amide, can be used in a proportion of 0.1 to 15% by weight relative to the total weight of the composition, and preferably from 1 to 10%. The sphingoid base content can vary according to the base used, but it is generally between 0.01% and 5%, preferably between 0.05 and 2%.

The nicotinic acid and the nicotinic acid amide, in particular vitamin PP, and also the sphingoid bases used in the compositions of the present invention are generally commercially available and can be obtained by known methods from various appropriate sources.

For example, the sphingoid bases can be obtained from natural sources or by chemical synthesis or by fermentation. They can also be obtained from animal or plant tissues by extraction and purification. Preferably, the spingoid bases used in the invention are prepared by microbial fermentation, for example from a yeast such as Pichia ciferii, and the phytosphingosine obtained in this way has the advantage of being very similar to that of human or animal skin. According to a preferred embodiment of the invention, phytosphingosine obtained from tetraacetylphytosphingosine by deacetylation is used as sphingoid base. The deacetylation reaction can be carried out by chemical reaction, for example by hydrolysis in a basic medium, or by enzymatic reaction.

The trials carried out on the use, according to the present invention, of the compositions described above have demonstrated a synergistic action most particularly between vitamin PP and phytosphingosine, providing a potentiation of the depigmenting activity.

Vitamin PP is thought to act mainly via a mechanism of inhibition of melanin transfer to the keratinocyte, whereas phytosphingosine is thought to inhibit NFkappaB translocation after exposure to UV radiation.

It is possible to incorporate into the compositions, the use of which is the subject of the present invention, another depigmenting agent, a list of examples of which was provided above in the introduction. It is also possible to combine exfoliants, and in particular lactic acid, with the compositions according to the present invention. All these composition variants are also part of the invention.

The excipients and carriers which can be used in the compositions in accordance with the present invention are those commonly used in preparations for cosmetic use, and are chosen according to the selected administration form. By way of example, mention may be made of gelling agents, emulsifiers, thickeners, preserving agents, softeners, and also fragrances.

The emulsifier can be chosen from high-molecular-weight carboxyvinyl polymers such as Carbopol®, polysorbates such as those sold under the trademarks Tween 20® or Tween 60®, sorbitan esters and, for example, a sorbitan stearate, palmitate, oleate or laurate (for example, Arlacel®). Emulsifiers which can be used also include various derivatives of stearic acid or palmitic acid, such as glyceryl stearate, a propylene glycol stearate, a polyethylene glycol stearate, PEG 100® stearate, a steareth or a ceteareth, or alternatively polyglyceryl-2-sesquioleate, polyoxyethylene cetyl ether, a siloxane polyglucoside, or an emulsifiable silicone.

The gelling agents and thickeners are incorporated into the composition so as to improve the fluidity thereof. They can be chosen, for example, from polyacrylamides of the carbopol type, acrylate/acrylic acid copolymers and, for example, Aculyn®, cellulose derivatives such as hydroxypropyl-cellulose and, for example, Klucel®, plant mucopoly-saccharides, waxes such as beeswax, clays or natural gums such as xanthan gum.

The softeners can be chosen from fatty alcohols or esters, and, for example, the products based on isostearyl alcohol or on polysaccharide sorbitol sold under the trademarks Soothex® and Rhamnosoft®. In general, the normal softeners of the art may be suitable in the invention.

Agents for protecting against ultraviolet rays can also be added to the compositions, the use of which is the subject of the present invention. These protecting agents may, for example, be hydrophilic or lipophilic UV-A and UV-B sunscreens which can be chosen from benzophenone or a benzophenone derivative such as 2-hydroxy-4-methoxybenzophenone (Eusolex® 4360), or an ester of cinnamic acid, and more particularly octyl methoxycinnamate (Eusolex® 2292), 2-ethylhexyl methoxy-cinnamate (Parsol MCX®), or else a cyano-β,β-diphenyl acrylate such as octocrylene (Eusolex® OCR), 4-methylbenzylidenecamphor (Eusolex 6300®), and dibenzoylmethane derivatives such as 4-isopropyldibenzoylmethane (Eusolex 8020), tert-butylmethoxy-dibenzoylmethane (Parsol 1789®) and 4-methoxydibenzoylmethane.

It is also possible to add to the compositions, the use of which is the subject of the present invention, pigments which form an anti-ultraviolet screen, and which can, for example, be chosen from titanium dioxide (amorphous or crystalline in rutile or anatase form), zinc oxide, zirconium oxide or else aluminum oxide. Use may in particular be made of nanopigments of metal oxides with a particle size of between 5 and 100 nm.

A solvent capable of facilitating or improving the penetration of the active ingredients into the skin can also be advantageously included in the compositions of the invention, and, for example, a nonionic, amphiphilic glycerol derivative such as 1,2-O-isopropylidene glycerol (Solketal) can be used.

The compositions for implementing the invention can be provided in any of the usual pharmaceutical forms suitable for the dermatological or cosmetological indication, for topical application.

They can be provided, for example, in the form of aqueous, oily or aqueous-alcoholic solutions, dispersions, sera, gels (aqueous, anhydrous or lipophilic), micellar lotions, aqueous-alcoholic lotions, solutions for spraying, suspensions, ionic or nonionic vesicular dispersions, liquid or semi-liquid emulsions (for example, a milk), or solid or semi-solid emulsions. The emulsions can be of the oil-in-water (O/W) or water-in-oil (W/O) type, for example gels or creams.

The compositions, the use of which is the subject of the present invention, can be applied directly to the skin at a rate of one or more applications a day for a period of time suitable for the duration of the condition. For example, in the case of the treatment of pigmentation spots of medium intensity in an individual 50 to 60 years old, good results can be obtained by applying a composition according to the invention twice a day for an uninterrupted period of 6 to 8 weeks.

The following examples illustrate the present invention without, however, limiting it.

### EXAMPLE 1

A blind study on volunteers was carried out in a sunny period (8 volunteers per product) . This study is based on an overall evaluation of the parameters of color of the dorsal skin of the hands on a mode “before” versus “after” use. The products were applied to the entire hand and the effect was evaluated on preselected pigmentary spots and on an area showing no spots (referred to as <<control>> site) . Each volunteer used a sun protection cream on the hands in order to minimize the risk of a hyperpigmentation liable to appear under the influence of the ultraviolet radiation related to the period of sunshine during the experiment.

The following parameters were studied
geometry of the spots (size),
melanin index on the spot and on the <<control>> site, and
luminance (L*) on the spot and on the <<control>> site.

The analysis relates to the evolution of the parameters between the first and last day of the study for the products applied.

The composition of the products studied is as follows:

| | Vitamin PP | Depigmenting agent |
|---|---|---|
| Formula 1 | 0% | 0% |
| Formula 2 | 5% | 0.1% salicyloyl phytosphingosine |
| Formula 3 | 5% | 0%% |
| Formula 4 | 0% | Arlatone dioic |
| Formula 5 | 0% | Albatin |

Thus, formula 1 is the placebo formula, formula 2 is based on the <<active base>> containing vitamin PP at the concentration of 5%, to which a sphingoid base has been added, while formula 3 contains the same dose of vitamin PP without phytosphingosine. Formulae 4 and 5 are commercially available compositions containing listed depigmenting agents, namely octadecene dioic acid (Arlatone dioic) and aminoethyl-phosphinic acid (Albatin).

The results obtained show that:
For the formulations 1, 2 and 3, the geometry of the spots does not significantly change over time.
The luminance (L*) of the skin increases (the color of the skin lightens) on the spots and on the <<control>> area for formula 2.
The melanin index decreases (less melanin is present) on the spot and on the <<control>> area for formula 2.

For the placebo formula (formula 1) and for formula 3 containing vitamin PP without phytosphingosine, over time, the melanin index notably increases, and in a comparable manner despite the use of the sun product. The same is true of formulae 4 and 5, which have no substantial effect on the melanin index and on the luminance. On the other hand, when formula 2 is used, a decrease in this melanin index is observed. The decrease observed is stronger on the pigmentary spots than on the <<control area>>.

It should also be underlined that 63% of the volunteers (5 out of 8) pointed out, for formula 2, a decrease in the color of the spots which is scored <<yes, clearly>>, and <<yes, probably>>.

Overall, this study confirms the advantage of the combination of vitamin PP and 0.1% salicyloyl phytosphingosine as a depigmenting agent.

In addition, a comparative study of the antityrosinase activity made it possible to demonstrate the potentiation of the depigmenting effect resulting from the combination of vitamin PP and phytosphingosine.

Antityrosinase activity is recognized as an in vitro indicator of the depigmenting activity of a composition.

The trials were carried out by comparing two known commercially available depigmenting formulae (A and B) and a formula in accordance with the invention (formula C):

Formula A: commercial composition (TRIO D®)

Formula B: commercial composition (TRIO A®)

Formula C: identical to Example 5 below.

TRIO D is a depigmenting composition based on ammonium lactate, ascorbic acid and glabridine at the concentration of 0.1%. TRIO A comprises the same active ingredients and glabridine at the concentration of 0.2%. Formula C of the invention contains the same concentration of flabridine, which is a compound known for its antityrosinase activity.

The trials were carried out according to conventional measuring techniques, the compositions being in an ethanolic solution buffered at pH 6.8 containing 500 μl of tyrosine solution and 500 μl of tyrosinase solution, and the control used is identical but contains no depigmenting agent. A control blank is identical to the control, but the tyrosinase is replaced with the same amount of water. Similarly, a trial blank is prepared using the same sample of formula to be tested, but replacing the tyrosinase with the same amount of water.

The compositions are left to incubate for one hour in a water bath at 37° C. in the dark and then the tubes containing the formulae tested are cooled on a bath of cold water at approximately 18° C. The absorbance of the solutions is measured at 475 nm in a 10 mm cuvette.

The results are given in the following table:

| Formula | Amount μg | % inhibition | Degree/1 mg |
|---|---|---|---|
| A | 984 | 14 | 16 |
| | 996 | 19 | |
| | 1996 | 32 | |
| B | 1036 | 43 | 48 |
| | 509 | 30 | |
| | 1019 | 44 | |
| C | 996 | 61 | 93 |
| | 499 | 62 | |

The average degree of inhibition for an amount of product of 1 mg was obtained by calculating the mean of the measurements for each formula (3 measurements per formula) related to an amount of product of 1 mg.

It is therefore noted that formula C according to the invention exhibits a notably higher antityrosinase activity than the reference formulae, resulting in a greater depigmenting capacity. It is in particular noted that the degree of inhibition is doubled compared with formula B, which contains the same amount of glabridine. This demonstrates the effect of the combination of vitamin PP and phytosphingosine on depigmentation.

The following examples relate to formulations containing a combination, the use of which is the subject of the present patent application. The names of the components are derived from the INCI nomenclature.

### EXAMPLE 2

### Depigmenting Emulsion

| | |
|---|---|
| Capric/caprylic triglyceride | 5.00% |
| PPG-15 stearyl ether | 5.00% |
| Arachidyl alcohol (and) behenyl alcohol | 3.00% |
| (and) arachidyl glucoside | |
| salicyloyl phytosphingosine | 0.10% |
| PEG-100 stearate (and) glyceryl stearate | 1.50% |
| Cetyl alcohol | 1.00% |
| Dimethicone | 1.00% |
| Water | qs 100 |
| Xanthan gum | 0.30% |
| Preserving agents | qs |
| Fragrance | qsp |
| Nicotinamide | 5.00% |
| Lactic acid | 5.00% |
| Base | qs pH = 4.5 |

### EXAMPLE 3

### Depigmenting Emulsion

| | |
|---|---|
| Capric/caprylic triglyceride | 5.00% |
| PPG-15 stearyl ether | 5.00% |
| Isopropyl palmitate | 4.00% |
| salicyloyl phytosphingosine | 0.10% |
| steareth-2 | 3.00% |
| steareth-21 | 2.00% |
| PEG-100 stearate (and) glyceryl stearate | 1.00% |
| Cetyl alcohol | 1.00% |
| Dimethicone | 1.00% |
| Water | qs 100 |
| Xanthan gum | 0.30% |
| Preserving agents | qs |
| Fragrance | qs |
| Glabridine | 0.20% |
| Nicotinamide | 5.00% |
| Undecylanoyl phenylalanine | 2.00% |
| Acid/base | qs pH = 4.5 |

### EXAMPLE 4

### Alcoholic Gel

| Alcohol | qs 100 |
|---|---|
| Cyclopentasiloxane | 13.00% |
| Propylene glycol | 16.00% |
| Nicotinamide | 4.00% |
| C12/15 alkyl benzoate | 12.00% |
| Glabridine | 0.20% |
| Salicyloyl phytosphingosine | 0.20% |

### EXAMPLE 5

### Solution

| | |
|---|---|
| Capric/caprylic triglyceride | 12.00% |
| Solketal | 48.00% |
| Phytosphingosine | 0.10% |
| Nicotinamide | 5.00% |
| Alcohol | qs 100 |

1. The use of a composition comprising a combination of at least nicotinic acid or a nicotinic acid amide and of at least one sphingoid base, as a depigmenting agent.

2. The use as claimed in claim 1, characterized in that the sphingoid base is chosen from sphingosine, sphinganine, phytosphingosine, salicyloyl phytosphingosine, phytosphingosine salicylate, tetracetylphytosphingosine, N-acetyl-phytosphingosine and phytosphingosine hydrochloride.

3. The use as claimed in claim 2, characterized in that the sphingoid base is chosen from phytosphingosine, salicyloyl phytosphingosine or phytosphingosine hydrochloride.

4. The use as claimed in claim 1, characterized in that the nicotinic acid amide is nicotinamide.

5. The use as claimed in claim 1, characterized in that the sphingoid base content in the composition is between 0.01% and 5% relative to the total weight of the composition.

6. The use as claimed in claim 5, characterized in that the sphingoid base content is between 0.05% and 2% relative to the total weight of the composition.

7. The use as claimed in claim 1, characterized in that the content of nicotinic acid, or of nicotinic acid amide, in the composition is between 0.1 and 15% by weight relative to the total weight of the composition.

8. The use as claimed in claim 7, characterized in that the content of nicotinic acid, or of nicotinic acid amide, in the composition is between 1 and 10% by weight relative to the total weight of the composition.

9. The use as claimed in claim 1, for promoting lightening of the skin.

10. The use as claimed in claim 1, for controlling hyperpigmentation.

11. A method for cosmetic treatment of the skin, intended to reduce, eliminate or avoid pigmentation spots, by providing a depigmenting effect, consisting in applying to the exposed areas a composition as claimed in claim 1.

12. The method for cosmetic treatment as claimed in claim 11, characterized in that it is intended to promote lightening of the skin.

13. The method for cosmetic treatment as claimed in claim 11, characterized in that it is intended to control skin hyperpigmentation.

14. The use of a composition as claimed in claim 1, for preparing a dermatological composition intended to control hyperpigmentation.
